(19) United States
(12) Patent Application Publication
(10) Pub. No.: US 2014/0323505 A1
(43) Pub. Date: Oct. 30, 2014

(54) ORAL RINSE COMPOSITION AND METHOD TO DELIVER ENERGY SUPPLEMENTS

(71) Applicant: Rinsenergy LLC, Colleyville, TX (US)

(72) Inventors: Howard Ketelson, Colleyville, TX (US); David Meadows, Colleyville, TX (US); Rekha Rangarajan, Colleyville, TX (US); Yen Tran, Colleyville, TX (US)

(73) Assignee: Rinsenergy LLC, Colleyville, TX (US)

(21) Appl. No.: 14/261,113

(22) Filed: Apr. 24, 2014

Related U.S. Application Data
(60) Provisional application No. 61/816,005, filed on Apr. 25, 2013.

(51) Int. Cl. A61K 9/08; A61K 31/355; A61K 45/06; A61K 31/522

(52) U.S. Cl. CPC A61K 9/08; A61K 31/522; A61K 31/355; A61K 45/06
USPC 514/263.34; 514/458

(57) ABSTRACT

An oral rinse composition that comprises an aqueous solution that includes a high molecular weight polymer and an energy supplement that stimulates central nervous system activity.

### CROSS-REFERENCE TO RELATED APPLICATION

This application claims the benefit of U.S. Provisional Application No. 61/816,005 filed by Howard Ketelson, et al., on Apr. 25, 2013, entitled “ORAL RINSE COMPOSITION AND METHOD TO DELIVER ENERGY SUPPLEMENTS,” commonly assigned with this application and fully incorporated herein by reference.

### TECHNICAL FIELD

This application is directed, in general, to oral rinses and, more specifically, to an oral rinse composition to deliver energy supplements and to methods of preparing such compositions.

### BACKGROUND

A large segment of the population currently consumes energy supplements for staying alert and improving performance in physical and mental activities. However, the market penetration of energy supplements may be limited due to several issues related to the delivery systems presently being used.

Often, the energy supplement is delivered in the form of a liquid drink, chewing gum, tablets dissolvable films, spray in the mouth, transdermal, and nasal spray. Liquid drinks can be expensive and challenging to carry around due to liquid weight. Liquids and tablets are swallowed and need to pass through the gastrointestinal tract for absorption and onset of action. This, in turn, can reduce the bioavailability of the energy supplement (e.g., due to the absorption competition with food (proteins, carbs, fats, fibers, etc. . . . ) and require a long duration (e.g., about 1 hour) or variable durations (e.g., due to the variable competition of absorption) for effects of the energy supplement to be felt. Tablets and gums can be challenging and expensive to manufacture. For instance, tablets need to be designed to dissolve and pass through the gut. In addition, tablet and gum delivery systems can pose a choking hazard, especially if the consumer is engaging in physical activity during consumption. Any of these delivery systems may have high concentrations of sugar, calories or the energy supplement itself. This, in turn, can cause certain segments of the population to not consume the energy supplement due to a dislike in the taste of the energy supplement, the delivery system or due to health concerns.

Accordingly, what is needed in the art is an energy supplements delivery system that does not suffer from the disadvantages associated with the conventional delivery systems discussed above.

### SUMMARY

To address the above-discussed deficiencies, the present disclosure provides in one embodiment, an oral rinse composition that comprises a high molecular weight polymer and an energy supplement.

In some embodiments, the energy supplement can include or be caffeine having a concentration in the aqueous solution in a range from about 0.001 to about 2% Wt/Vol. In some such embodiments, the caffeine concentration in the aqueous solution can be in a range from about 0.2 to about 1% Wt/Vol. In any such embodiments, the energy supplement can include one or more of an amino acid, a phospholipid, a vitamin, a cellular metabolite, or an acetyl choline enhancer. In some embodiments, the high molecular weight polymer can have a molecular weight of at least about 100,000 grams per mole and less than about 7 million grams per mole. In some such embodiments, the molecular weight can be in a range from about 1 to about 3 million grams per mole. In some embodiments, the oral rinse composition at room temperature and zero shear, can have a viscosity in the range from about 4 cp to about 10 cp. In some such embodiments, at a shear rate in a range from about 50 sec⁻¹ to about 1000 sec⁻¹ the viscosity of the oral rinse composition can drop by at least about 10 percent within about 30 seconds, and, the viscosity of the oral rinse composition can return to the viscosity at zero shear value upon terminating of the shear rate in the range. In some embodiments, the high molecular weight polymer can include or be a poly (ethylene oxide) polymer. In some embodiments, the high molecular weight polymer can have a concentration in the aqueous solution that is in a range from about 0.01 to about 10 wt/vol %. In some embodiments, the high molecular weight polymer can include or be a hydrophilic polymer. In some embodiments the oral rinse composition can include a buffering agent and has a pH in a range from about 3 to about 8. In some embodiments the oral rinse composition can include one or more of a surfactant, a skin and mucosal penetration enhancer, a cooling agent, a warming agent or a flavoring agent. In some embodiments, the oral rinse composition is an emulsion of the aqueous solution and a water-insoluble one of a flavoring agent, cooling agent, warming agent or stabilizer.

In another embodiment, the oral rinse composition includes or is an aqueous solution that includes: caffeine, sodium citrate and citric acid, menthyl glycerin acetal, polysorbate 20, spearmint oil, peppermint oil, vitamin E, Xylitol, monk fruit extract, pH equal to about 6.5 and the high molecular weight polymer includes a poly(ethylene oxide) polymer having a molecular weight of about 1 million.

Still another embodiment of the disclosure is a method of manufacturing an oral rinse composition. The method comprises preparing an aqueous solution, including: providing a high molecular weight polymer, providing an energy supplement that stimulates central nervous system activity and dissolving the high molecular weight polymer and the energy supplement in water to form an aqueous solution.

Some embodiments of the method can further include preparing an aqueous dispersion and mixing the aqueous solution and the aqueous dispersion together to form an aqueous mixture such that the flavoring agent and surfactant are uniformly dispersed throughout the aqueous mixture. In some embodiments, preparing an aqueous dispersion can include providing a flavoring agent, providing a surfactant and mixing the flavoring agent and surfactant together in water such that the flavoring agent and surfactant are uniformly dispersed in the water.

Some embodiments of the method can include adding acid or base to adjust the aqueous solution to provide a pH in a range from about 3 to about 8. Some embodiments of the method further include sterilizing the aqueous solution. Some embodiments of the method can further include further including filtering the aqueous solution to provide a clear oral rinse composition.

### BRIEF DESCRIPTION

For a more complete understanding of the present disclosure, reference is now made to the following detailed description taken in conjunction with the accompanying FIGUREs. It is emphasized that various features may not be drawn to scale. In fact, the dimensions of various features may be arbitrarily increased or reduced for clarity of discussion. Reference is now made to the following descriptions taken in conjunction with the accompanying drawings, in which:

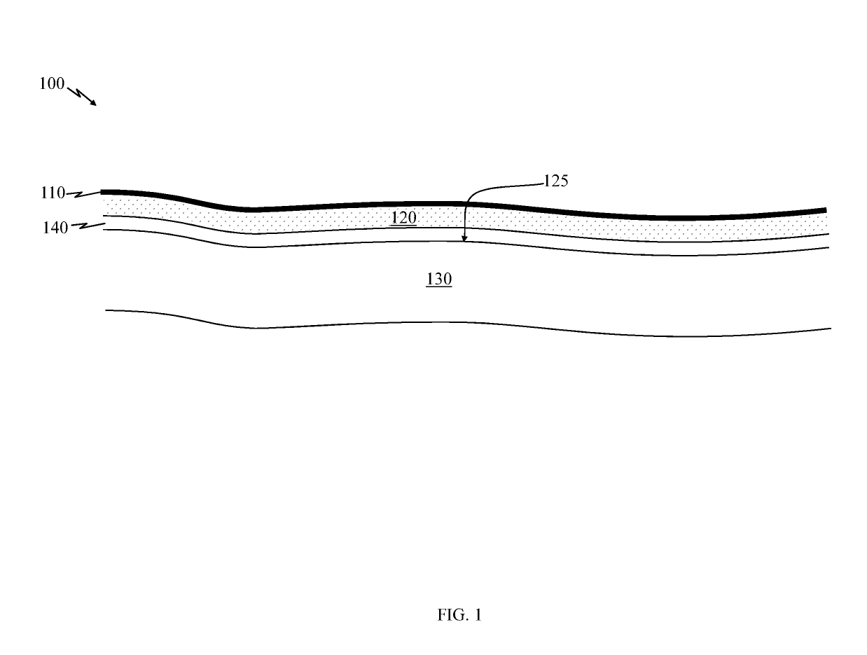

FIG. 1 presents a cross-sectional view of an exemplary residual oral rinse composition of the present disclosure after delivery to the oral cavity of a human subject;

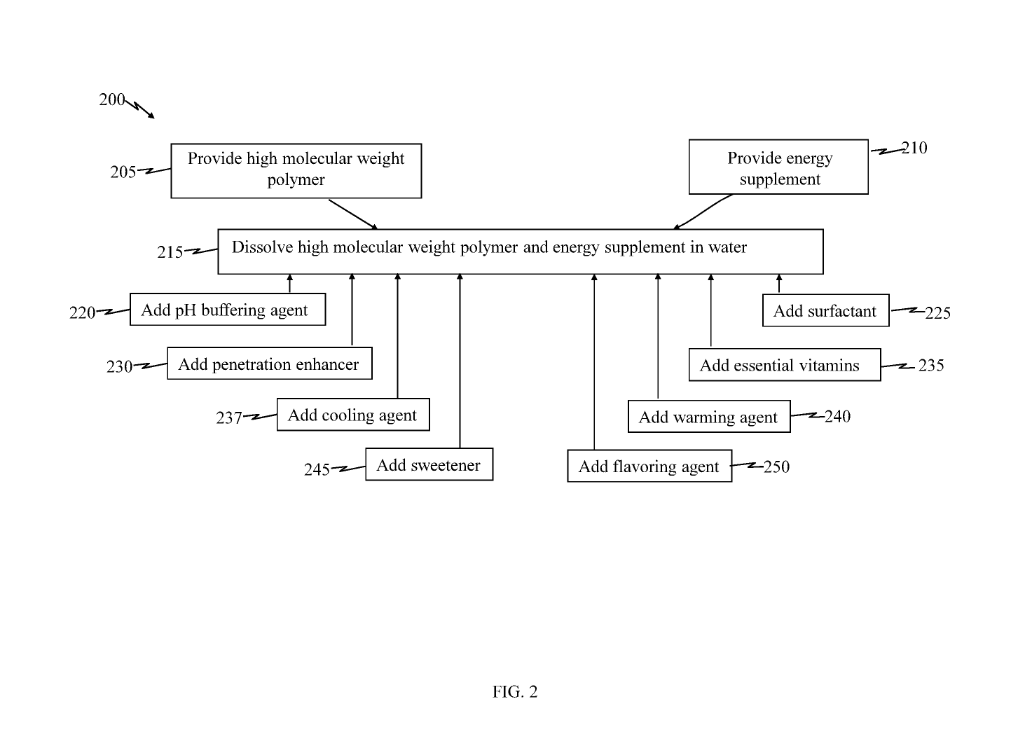

FIG. 2 illustrates by flow diagram, selected aspects of an example method of preparing an oral rinse composition according to the principles of the present disclosure; and

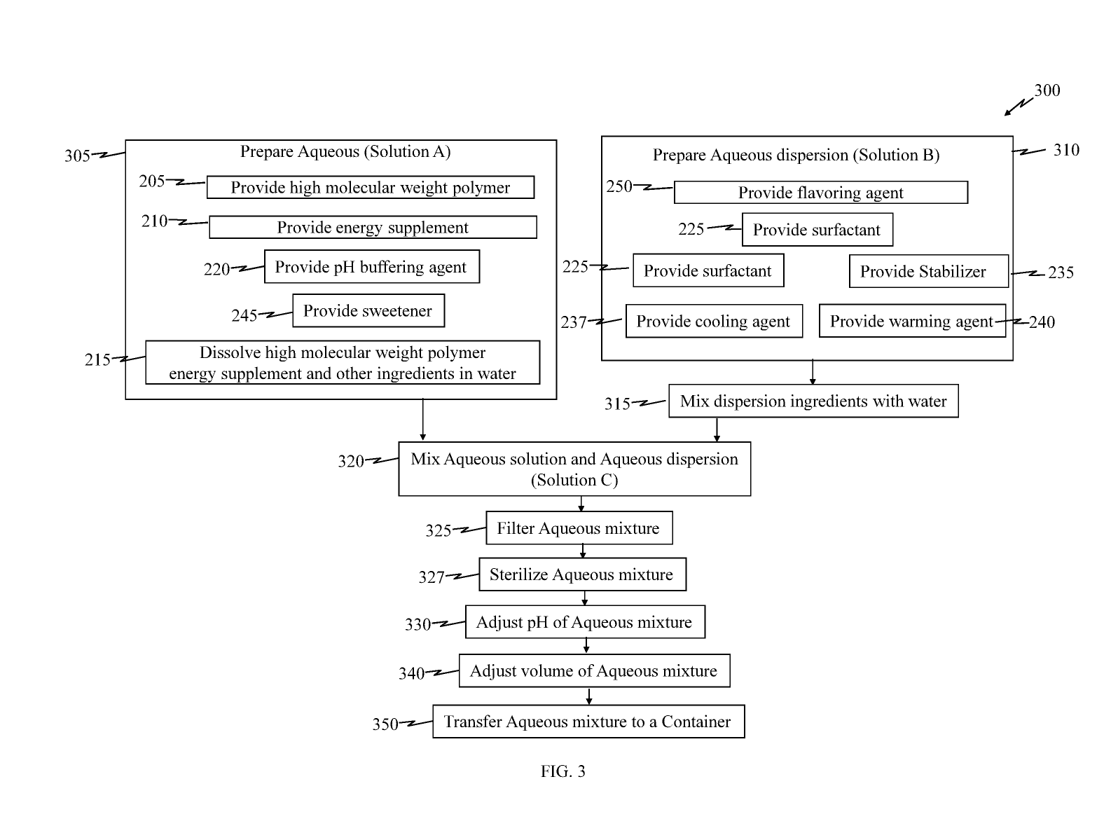

FIG. 3 illustrates by flow diagram, selected aspects of another example method of preparing an oral rinse composition according to the principles of the present disclosure.

### DETAILED DESCRIPTION

Embodiments of the present disclosure benefit from the recognition that an oral rinse composition can provide an effective and efficient route to deliver energy supplements directly into the blood stream via absorption of energy supplements into various tissues of the oral cavity including any or all of the cheeks, tongue, palates, glands, lips, gums, or tonsils. As part of the present disclosure, it was discovered that the absorption of energy supplements into these tissues is enhanced by including in the oral rinse, a high molecular weight polymer.

While not limiting the scope of the disclosure by theoretical considerations, it is believed that including a high molecular weight polymer in the oral rinse helps to retain the energy supplement in close proximity to the tissues of the oral cavity and thereby facilitates increased absorption of the energy supplement into these tissues.

FIG. 1 presents a cross-sectional view of an exemplary residual oral rinse composition after delivery to the oral cavity of a human subject. It is believed that after the oral rinse composition has been swirled in the mouth and the bulk of it has been expectorated, the high molecular weight polymer present in the portions of the oral rinse retained in the mouth can form a highly viscous film 110. It is believed that the highly viscous film 110 acts as a mechanical barrier that holds the retained portions of the energy supplement 120 in between the high-viscosity film 110 and a surfaces 125 of the oral cavity tissues 130, thereby facilitating increased absorption of the energy supplement into the tissue.

As part of the present disclosure, it was discovered that the molecular weight, the viscosity, and thixotropic properties of the high molecular weight polymers disclosed herein are important variables that affect the efficacy of the oral absorption of the energy supplement. That is, one or more of the ranges of molecular weights, viscosity, or shear profiles of the polymers disclosed herein are newly discovered result-effective variables that influence the oral tissue absorption of energy supplements or other ingredients as further discussed below.

The term, energy supplement, as used herein refers to any naturally occurring ingredient that acts as a central nervous system stimulant in humans. For instance, in some cases, a human subject taking the energy supplement can perceive increased mental energy, alertness or focus. For instance, in some cases, a human subject taking the energy supplement has increased brain activity (e.g., at least about a 10 percent increase in activity, as measured by EEG, PET, fMRI or similar procedures familiar to those skilled in the pertinent arts) and neurotransmission e.g., by enhancing the release of neurotransmitters.

In some embodiments of the oral rinse composition, the energy supplement includes, or is, caffeine, or similar compounds acting as an adenosine antagonist in the brain, thereby causing increased neurotransmission and giving the human subject a sensation of increased energy or focus. Without limitation the caffeine can be sourced from coffee beans, Cocoa nut, Guarana, Yerba mate, or Kola nut.

Non-limiting examples of other energy supplements include compounds in the categories of amino acids such as L-tyrosine, L-taurine, L-phenylalanine, L-Theanine and L-glutamine, phospholipids, such as phosphatidylserines (PS), phosphatidylcholines (PC), or Lecithin, or vitamins such as Vitamin C, Vitamin D, B Vitamins, including Vitamin B6 and Vitamin B12, or Vitamin E. Other examples of energy supplements include cellular ingredients such as malic acid, glucuronolactone, CoQlO, ATP, Creatine monohydrate. Still other examples of energy supplements include acetyl choline enhancers such as Acetyl L Carnitine, Dimethyl aminoethane, Fisetin, Huperzine, Citicoline, Choline. Yet other examples of energy supplements include homeopathic ingredients such as ephedra, herbal extracts used in traditional ayurvedic texts as energy boosters, such as Ashwagandha, Shilajit, Licorice root, Gotu kola, or other natural compounds such as quercetin, grape seed extract, Acai berry, Catechins, Flavonoids, or Spirulina.

Although a number of example formulations disclosed herein feature the use of caffeine as the energy supplement, any of the above-described energy supplements can be used as an alternative, or in addition, to caffeine as the energy supplement.

The term, high molecular weight polymer, as used here refers to a polymer has a molecular weight of at least about 100,000 grams per mole (g/mol). The use of polymers having a molecular weight of less than about 100,000 g/mol are not expected to have the desired viscosity characteristics to provide an effective barrier that holds the energy supplement in proximity to the tissues in the mouth, and thereby promote delivery of the energy supplement into the bloodstream.

In some embodiments, the high molecular weight polymer has a molecular weight of less than about 7 million g/mol. The use of polymers of greater than about 7 million g/mol are less desirable because the viscosity of such polymers is so high as to feel uncomfortable in the mouth, e.g., when swirling the disclosed oral rinse composition containing such polymers in the mouth.

In some embodiments, the high molecular weight polymer has a molecular weight in a range from about 1 to about 3 million g/mol. The use of high molecular weight polymers in this molecular weight range is expected to provide the desirable combination of forming a high-viscosity film adjacent to the tissues in the mouth and not being readily de-adsorbed from the surfaces of the oral tissues, while at the same time, not having so high a viscosity as to be feel uncomfortable in the mouth or difficult to swirl the disclosed oral rinse compositions containing such polymers in the mouth.

In some embodiments, the high molecular weight polymer in the oral rinse composition at room temperature and zero shear (e.g., a shear rate of less than or equal to 1 s⁻¹), has a viscosity in the range from about 1 centipoise (cp) to about 50 cp, and more preferably from about 1 cp to about 10 cp. Such viscosity ranges help to provide the desired viscous properties of residual oral rinse composition in the mouth, after expectoration, so as to form the high-viscosity film of high molecular weight polymer.

In some embodiments, the high molecular weight polymer in the oral rinse composition has thixotropic properties at shear rates typically found in the mouth, e.g., between about 50 sec⁻¹ to 1000 s⁻¹ to promote distributing the high molecular weight polymer and to form the high-viscosity layer. For instance, it is desirable for the viscosity of the oral rinse composition to drop quickly as it is swirled around in the mouth. Then, after expectoration of the oral rinse, it is desirable for the viscosity of the retained portion of the oral rinse composition to rapidly (e.g., within 5 to 10 seconds) increase as the mouth enters a static (e.g., non-swirling, non-shearing) state so as to promote formation of the high-viscosity film.

While not limiting the scope of the disclosure by theoretical considerations, it is believed that the oral rinse can serve as polymer delivery system that is designed to behave as a viscous fluid in the mouth with desirable sensory attributes (lubricacious, non-tacky, shear thinning at high shear rates). The viscous fluid should shear thin at shear rates similar to those occurring in the mouth and on the oral surface. It is believed that shear rates in a range from about 50 s⁻¹ to about 1000 s⁻¹ are important for shear thinning characteristics in the mouth especially when swirling a rinse in the mouth. Following the swirling and expectorating liquid from mouth the polymer retained in the mouth should return quickly back to its original high viscosity as a film. While not limiting the disclosed embodiments by theory, it is believed that this quick return to the original high viscosity is facilitated by the high molecular weight polymers having non-viscoelastic properties, that is, thixotropic properties. For instance, at least for some embodiments, it is believed that this effect is facilitated when the polymer solution has a minimal elasticity under reduced shearing action in the mouth.

Surprisingly, it has been discovered that the use of specific polymers as disclosed herein have these desired viscosity properties for a rinse delivery technology.

Table 1 below presents example viscosity data as a function of shear rates for formulations for one water soluble poly (ethylene oxide) polymer having a molecular weight of about million (e.g., WSK N-12, POLYOX, DOW; a number average molecular weight about 1 million±10 percent). Concentration ranges of about 0.2%, 0.6% and 1.0% (wt/vol %) are provided as examples. The about 0.2% and 0.6% concentrations show shear thinning starting at approximately 60 sec⁻¹ with greater magnitudes in shearing occurring closer to 1000 sec⁻¹. The 0.1% concentration shows shear thinning at much lower shear rates and behaves more as a thixotropic solution. The 0.2% and 0.6% concentrations have viscosities that match more closely with those shear rates occurring in the mouth during rinsing.

TABLE 1
Table 1: Table of example viscosity data for POLYOX

| 0.2% Polyox Example | | 0.6% Polyox example | | 1.0% Polyox example | |
|---|---|---|---|---|---|
| Shear rate | Viscosity | Shear rate | Viscosity | Shear rate | Viscosity |
| (sec⁻¹) | (cp) | (sec⁻¹) | (cp) | (sec⁻¹) | (cp) |
| 6.309 | 3.693 | 6.46 | 12.98 | 6.312 | 32.89 |
| 9.999 | 3.69 | 10.144 | 12.98 | 12.44 | 32.67 |
| 63.096 | 3.67 | 62.750 | 12.65 | 64.72 | 30.29 |
| 99.999 | 3.66 | 100.259 | 12.40 | 106.544 | 28.81 |
| 158.499 | 3.64 | 162.128 | 12.03 | 178.746 | 26.89 |
| 251.189 | 3.62 | 264.574 | 11.54 | 307.12 | 24.56 |
| 398.106 | 3.58 | 436.605 | 10.95 | 536.898 | 21.99 |
| 630.958 | 3.52 | 730.327 | 10.269 | 713.203 | 20.69 |
| — | — | 948.41 | 9.903 | 953.198 | 19.39 |
| — | — | 1231.41 | 9.531 | 1277.66 | 18.11 |

In some embodiments, the high molecular weight polymer is a hydrophilic polymer. The term hydrophilic polymer as used here refers to a polymer having very high, and in some cases, infinitely high (e.g., full miscibility), solubility in water. Such hydrophilic high molecular weight polymers have various hydrogen bonding groups, such as ethylene oxide, as part of the primary polymer chain and/or as side chains. Other non-limiting examples of such hydrogen bonding groups include hydroxide, or carboxylate, groups.

In some embodiments the use of a hydrophilic high molecular weight polymers is desirable because such hydrophilic polymers can adhere to a glycocalyx layer 140 (FIG. 1) covering the oral cavity tissues 130, thereby promoting formation and retention of the high-viscosity film. For instance, while not limiting the scope of the disclosure by theory, it is believed that in some cases, the hydrogen bonding groups of the hydrophilic high molecular weight polymers of the disclosure can form hydrogen bonds with molecules present in the glycocalyx layer. For instance, in some cases it is believed that the hydrophilic high molecular weight polymers of the disclosure can form hydrogen bonds with the proteins of mucins (e.g., mucoproteins, glycoproteins, glycosaminoglycans, and glycolipids) covering the oral cavity tissues.

The hydrophilic high molecular weight polymers of the disclosure are surprisingly effective at enhancing absorption of the certain subset of energy supplements or other ingredients (e.g., cooling agents) having non-polar or hydrophobic properties having low water solubility. For instance, it was surprising that the disclosed hydrophilic high molecular weight polymers were effective at holding low water solubility energy supplements, such as caffeine, in the oral cavity for long periods (e.g., about 30 to 40 minutes). One might expect such low water solubility energy supplements to not strongly associate with a hydrophilic molecule and therefore be rapidly dissipated from the mouth. However, as discussed above, the hydrophilic high molecular weight polymers are believed to promote forming the high-viscosity film with a long residence time in the mouth (e.g., at least about 30 minutes, and in some cases, at least about 60 minutes and in some cases from about 30 to 60 minutes). This, in turn, is thought to provide a long-lasting mechanical barrier that helps to keep the low water solubility energy supplements or other ingredients in proximity to the tissues of the oral cavity, thereby promoting adsorption of the energy supplement into the tissues of the oral cavity.

Non-limiting examples of the hydrophilic high molecular weight polymers of the disclosure include mucosal adhesive polymers such as polyethylene oxide, also referred to herein as polyox.

In some embodiments of the oral rinse composition, the high molecular weight polymer is provided in sufficient amount to promote the formation of the high-viscosity film substantially over all of the soft tissue surfaces of the oral cavity (e.g., about 170 cm²) and thereby facilitate maximum absorption of the energy supplement into the tissue. For instance, in some embodiments, the concentration of the high molecular weight polymer in the oral rinse composition is in a range from about 0.01 to about 10 weight per volume percent (% Wt/Vol), and in some cases from about 0.2 to about 0.6% Wt/Vol.

In some embodiments, the oral rinse composition, at room temperature and zero shear, has a viscosity in a range of about 4 to about 10 cp, and in some embodiments, about 5 to about 9 cp, and in some embodiments, about 7 cp (e.g., 7±1 cp, or 7±0.5 cp). In some embodiments, at a shear rate in a range from about 50 sec⁻¹ to about 1000 sec⁻¹, the viscosity of the oral rinse composition drops by at least about 10 percent (and in some embodiments at least about 20 percent) within about 30 seconds, and, the viscosity of the oral rinse composition returns to the viscosity at zero shear value upon terminating of the shear rate in the range.

In some embodiments, the oral rinse composition can have a surprising lower dosage of the energy supplement, as compared to the dosage provided in conventional delivery systems, and yet still provide rapidly acting stimulant effect.

As a non-limiting example, consider the case of the energy supplement being caffeine. In some energy supplement drinks, such as coffee, tea or cola, typically contain about from 15 to 180 mg of caffeine per 150 to 180 ml of fluid per drink. Orally ingested caffeine thought to be absorbed by the small intestine within about 45 minutes of ingestion and then distributed throughout all tissues of the body with a peak blood concentration being reached within about one hour.

In contrast, in some embodiments, of the caffeine dosage in the oral rinse composition can range from about 0.001 to about 2% Wt/Vol of caffeine, or equivalently, from about 0.01 mg/ml to about 20 mg/ml. Thus, for example, the total dosage of caffeine in an oral rinse composition of 15 ml can be as low as 0.15 mg caffeine, which is about 100 to 1200 times smaller than the total dosage of caffeine present in some conventional drinks. And, as only about 1 ml of oral rinse composition remains in the mouth after expectoration, the dosage of caffeine available for absorption is smaller. For instance, continuing with the same example, the residual caffeine in the mouth available for absorption into the tissues or the oral cavity can be in the range of from about 0.01 mg to about 20 mg. Because the caffeine from the oral rinse provides for rapid absorption, it is thought that there can be increased bioavailability and a faster onset of stimulation as compared to to oral ingestion of a drink. For instance, the direct absorption of caffeine via the tissue in the oral cavity is thought to efficiently (e.g., rapidly) deliver optimal concentrations of the energy supplement.

In some embodiments, it is preferable for the oral rinse composition to include caffeine in a concentration range from about 0.2 to about 1% Wt/Vol, and in some cases, from about 0.2 to about 0.6% Wt/Vol. Such concentration range is believed to provides an amount of caffeine to produce a substantial stimulation but still remain well-below the aqueous solubility limit of caffeine (e.g., about 2% Wt/Vol at room temperature). Such a concentration range also is believed to provide a safe dosage of caffeine and to promote the stability the caffeine in the oral rinse composition.

In some embodiments, it is preferable for the oral rinse composition to have a pH of from about 3 to about 8, and more preferable from about 5 to about 8, and more preferably from about 6.1 to about 7.5, and in some cases about 6.5 (e.g., 6.5±0.2). More acidic pH values lower than this range are thought to promote degradation of the high molecular weight polymer. More alkaline pH values higher than this range can cause the oral rinse to have an undesirable soapy characteristic. In some embodiments, to facilitate providing a stabile pH in such ranges, the oral rinse further includes a buffering agent such as, but not limited to, more or more of citric acid and citrate, phosphoric acid, boric acid, ascorbic acid or combinations thereof.

In some embodiments to reduce its soapy characteristic, the oral rinse composition includes a surfactant such as, but not limited to, one or more of polysorbate 20 or polysorbate 80, in a concentration range of from about 0.01 to about 10% Wt/Vol.

In some embodiments, to enhance the absorption of caffeine into the tissues of the oral cavity, the oral rinse composition further includes one or more skin and/or mucosal penetration enhancer such as, but not limited to, one or more of fatty acids or cyclodextrins or similar penetration enhancer familiar to those skilled in the pertinent arts. In some embodiments the concentration of such penetration enhancers in the oral rinse is in the range from about 0.001 to about 10% Wt/Vol.

In some embodiments, the oral rinse composition may include one or more additional, or alternative, ingredients 145. Similar to that discussed above for the energy supplement 120, and not to be bound by theory, it is believed that the highly viscous film 110 can act as a mechanical barrier that holds the retained portions of the additional or alternative ingredients 145 in between the high-viscosity film 110 and a surfaces 125 of the oral cavity tissues 130, thereby facilitating increased absorption of the ingredient 145 into the tissue and providing longer-lasting effects as compared to presenting the ingredient 145 in an oral rinse with no high-molecular weight polymer (and resulting highly viscous film 110) present. Non-limiting examples of the ingredients 145 include compounds to freshening mouth breath and alleviating dry mouth symptoms, deliver vitamins, other health supplements, other drug actives or other compounds, include preservatives, colorants, alcohol, antiseptics, or anticavity compounds (e.g., like sodium fluoride).

For instance, in some cases, the oral rinse composition includes one or more essential vitamins at a concentration level that is soluble in an aqueous solution of the oral rinse and used between an amount effective to provide the minimum Recommended Daily Allowance, but, below the established tolerable upper limit or adequate intake level of said vitamin in an aqueous solution. Non-limiting examples of such vitamins include one or more of fat soluble vitamins, such as Vitamin A, Vitamin D, Vitamin E or Vitamin K, and water soluble vitamins, such as ascorbic acid, thiamine, riboflavin, niacin, pyridoxine, pantothenic acid, folic acid, cyanocobalamin, minerals or omega-3 FA.

For instance, in some embodiments, the oral rinse composition includes energy supplements such as one or more of amino acids, cellular ingredients, fatty acids, acetyl choline enhancers and other plant-based extracts or components. In some embodiments, the concentration of such energy supplements in a range from about 0.0001 and about 5% Wt/Vol.

In some embodiments, the oral rinse composition includes a cooling agent, that is, an ingredient that gives the human subject using the oral rinse with an oral cooling sensation. In some cases the cooling sensation can be longer-lasting (e.g., at least about 30 minutes, and in some cases, at least about 60 minutes, and in some cases, from 30 to 60 minutes) than if the cooling agent is in an oral rinse with no high molecular weight polymer present (e.g., a cooling sensation of several minutes). As a non-limiting example, in some embodiments the cooling agent includes a compound in the class of Transient receptor potential cation channel subfamily M member 8 (TRPM8) agonists capable of being effectively retained in the oral tissue with the high molecular weight polymer and slowly absorbed through the mucus membrane and thereby providing long-lasting cooling. In some such embodiments, the TRPM8 agonist is provided in a concentration range of from about 0.0001 to about 2% Wt/Vol, and in some cases, more preferably 0.4% Wt/Vol. In some embodiments, the cooling agent includes one or more of menthol, menth menthyl lactate (MML), or menthol acetyl, or menthyl glycerin acetal (MGA; e.g., Frescolat® menthyl glycerin acetal).

In some embodiments, the oral rinse composition includes a warming agent, that is, an ingredient that gives the human subject using the oral rinse with an oral warming sensation. As a non-limiting example, in some embodiments, the warming agent includes a compound in the class of transient receptor potential cation channel subfamily V member (TRPV) agonists, or, transient receptor potential ankyrin, member (TRPA) agonists. Non-limiting examples of such TRPV or TRPA agonists include one or more of Capsaicin, Piperine, Vanillyl butyl ether, or vanillyl ethyl ether. In some embodiments, the TRPV or TRPA agonist is provided in a concentration range of from about 0.0001 to about 2% Wt/Vol. In some embodiments, the warming agent can be co-delivered with one or more of the cooling agents listed above.

Some embodiments of the oral rinse composition includes a sweetener. In some cases the inclusion of a sweetener can facilitate masking of the undesirable taste of some energy supplements such as caffeine. Non-limiting examples of sweeteners include one or more of xylitol, Stevia extract, Monk fruit concentrate, Coconut sugar, Oat Sugar, pentadin, brazzein, agave, or honey. In some embodiments, the sweetener is provided in a concentration range of from about 0.001 to about 15% Wt/Vol and can include a combination of more than one type of sweetener.

Some embodiments of the oral rinse composition includes a natural or artificial flavoring agents, e.g., to help mask of the undesirable taste of some energy supplements. In some embodiments the flavoring agent can also advantageously serve as an antimicrobial agent to thereby prevent the growth of bacterial and yeast. Non-limiting examples of such flavoring agents include one or more of vanillin, wintergreen, peppermint oil, orange oil, spearmint oil, lemon oil, licorice, Cardamom, Ginger, Cinnamon, Clove, Fennel, Caraway, Grapefruit, Camphor, sassafras, natural and artificial cherry flavor, artificial champagne flavor, natural vanilla extract, ethylene vanillin, coffee extract, chocolate extract, artificial chocolate flavoring or cocoa extract. In some embodiments the flavoring agent can be botanical extracts (KESITLANT Plant, Drummondville, Canada) produced using a fermentation process using naturally occurring symbiosis of bacteria and yeast.

In some embodiments, the flavoring agent includes spearmint oil in Tween 20 in a 1:5 to 1:6 weight ratio added to an aqueous volume of the oral composition in a ratio of about 1:10. For example, in some cases, the oral rinse composition includes about 0.6 gm of spearmint oil added about 3 gm Tween 20, which in turn, is added to 33 gm of water. Such compositions desirably provide a clear solution of the oral rinse composition.

In some embodiments, the flavoring agent includes grapefruit oil and peppermint oil in Tween 20 in weight ratios of about 1:2.5:46. For example, in some cases, the oral rinse composition includes about 0.13 gm of grapefruit oil and 0.33 gm of peppermint oil added to about 5.97 gm Tween 20, which in turn, is added to 33 gm of water. Such compositions desirably provide a clear solution of the oral rinse composition.

In some embodiments, the flavoring agent includes 0.2 g of clove oil in 2 g of Tween-20, quantum satis to 20 mL with water. Such compositions desirably provide no discernible taste.

In some embodiments, the flavoring agent includes 0.2 g of ginger oil in 2 g of Tween-20, quantum satis to 20 mL with water. Such compositions desirably provide an energizing spicy taste.

In some embodiments, the flavoring agent includes 0.1 g of cinnamon oil in 2 g of Tween-20, quantum satis to 20 mL with water. Such compositions desirably provide a fresh sweet after-taste.

In some embodiments, the flavoring agent includes 0.1 g of cardamom oil, 0.2 g of cinnamon oil in 2 g of Tween-20, and 2.5 g of surfactant quantum satis to 20 mL with water. Such compositions desirably provide a clear solution having a refreshing long-lasting taste with a cool sensation.

In some embodiments, the flavoring agent includes 0.1 g of lemon oil, 0.1 g of ginger oil, in 3 g of Tween-20, quantum satis to 20 mL with water. Such compositions desirably provide a refreshing long lasting feeling.

In some embodiments, the flavoring agent includes 0.1 g of cinnamon, 0.1 g of lemongrass, in 2.5 g of Tween-20, quantum satis to 20 mL with water. Such compositions desirably provide an energizing feeling with a sweet after-taste.

As indicated, in some embodiments, the oral rinse composition comprises an aqueous solution that includes a high molecular weight polymer and an energy supplement that stimulates central nervous system activity. In some embodiments, the oral rinse composition can consist essentially of an aqueous solution that includes a high molecular weight polymer and an energy supplement that stimulates central nervous system activity. In some embodiments, the oral rinse composition can consist essentially of an aqueous solution having caffeine, sodium citrate and citric acid, menthyl glycerin acetal, polysorbate 20, spearmint oil, peppermint oil, vitamin E, Xylitol, monk fruit extract, pH equal to about 6.5 and the high molecular weight polymer includes a poly(ethylene oxide) polymer having a molecular weight of about 1 million. That is, in such embodiments, the oral rinse composition does not include other ingredients that would form or substantially contribute to forming the viscous film 110, or, would have substantial stimulating effect on central nervous system activity. Other embodiments of the oral rinse composition can further include other ingredients that synergistically promote forming the viscous film 110 such as: combinations of different molecular species of polymers, micro- and nano-emulsions, colloidal suspensions, pH, temperature, ion sensitive polymers, or metal ion (e.g., boron) crosslinking polymer systems.

Still another embodiment of the disclosure is a method of preparing an oral rinse composition that comprises a high molecular weight polymer and an energy supplement.

FIG. 2 illustrates by flow diagram, selected aspects of an example method 200 of preparing or manufacturing an oral rinse composition according to the principles of the present disclosure. Any of the above-described embodiments of the oral rinse composition can be prepared according to the method 200.

The method 200 comprises a step 205 of providing a high molecular weight polymer and a step 210 of providing an energy supplement. As a non-limiting example, in some embodiments, as part of step 205, about 0.4 grams of Polyox N12K is provided as the high molecular weight polymer. In some embodiments as part of step 210 0.2 grams caffeine is provided as the energy supplement. In some embodiments of the method, in step 215, the high molecular weight polymer and energy supplement provided in steps 205 and 210 respectively, are dissolved in water. As a non-limiting example, in some embodiments, as part of step 215, 0.2 grams of Polyox N12K and 0.2 grams caffeine are dissolved in about 100 ml of water. In some embodiments, about 0.6 grams of caffeine are dissolved in 100 mL of water with up to about 0.6 g of Polyox N12K. In some embodiments as part of step 215 the water is heated above room temperature (e.g., about 80° C.) to facilitate rapid dissolving of the high molecular weight polymer and the energy supplement in water. Some embodiments of the method 200 further include a step 220 of adding a pH buffering agent. As a non-limiting example, in some embodiments, as part of step 220, about 0.53 grams of sodium citrate and about 0.53 to 0.053 grams of citric acid are added to the about 100 ml of water used in step 215.

Although in the above example, the oral rinse composition is formulated in step 215 as an aqueous solution, other formulations of the oral rinse composition include emulsions or vesicle formats.

Some embodiments of the method 200 can further include, without limitation, any one or any combinations of ingredients such as: adding a surfactant (step 225), adding a penetration enhancer (step 230), adding essential vitamins (step 235), adding a cooling agent (step 237), adding a warming agent (step 240), adding a sweetener (step 245), or adding a flavoring agent (step 250). In some cases as part of step 250 the flavoring agent is prepared as a separate solution which is then combined with a solution that includes the other ingredients of the oral rinse composition.

To illustrate further aspects of the method FIG. 3 presents by flow diagram, aspects of another example method 300 of preparing an oral rinse composition according to the principles of the present disclosure. With continuing reference to FIG. 2, the method 300 comprises a step 305 of preparing an aqueous solution (e.g., Solution “A” as referred to in FIG. 3 and elsewhere herein). Preparing an aqueous solution (step 305 can include steps 205, 210 of providing a high molecular weight polymer (e.g., polyox N12K WSR) and providing an energy supplement (e.g., caffeine), respectively, and, a step 215 of dissolving the high molecular weight polymer and energy supplement in water to form the aqueous solution of the oral rinse of the oral rinse composition. In some embodiments, as part of step 215, the aqueous solution is heated in a range from to 60 to 80° C., and in some embodiments about 70° C. to facilitate the rapid dissolution of the oral rinse components in water. In some embodiments, as part of step 215, solid amounts of the high molecular weight polymer, energy supplement and additional optional constituents, further described below, are slowly added to water (at room temperature, and in some embodiments heated) with controlled speed mixing to mitigate foaming of the solution and clumping of protein. In some embodiments each of the ingredients are sequentially and separately added to facilitate dissolution.

As a non-limiting example, in some embodiments in step 305, purified water, equivalent to 80% of the final batch weight of the oral rinse composition, is added to a mixing vessel equipped with a suitable agitator. The water is heated to 70° C. Next the following ingredients, in the order listed, are slowly added allowing each ingredient to completely dissolve in the water before adding the next ingredient: Sodium Citrate; Citric Acid; Xylitol; Caffeine; Monk Fruit Extract; and Polyox WSR.

Some embodiments of the method 300 further include a step 310 of preparing an aqueous dispersion (e.g., Solution “B” as referred to in FIG. 3 and elsewhere herein).

In some embodiments of the method 300, preparing the aqueous dispersion (step 310) includes providing a flavoring agent (step 250), providing a surfactant (step 225) and a step 315 of mixing the flavoring agent and surfactant together in water such that the flavoring agent and surfactant are uniformly dispersed in the water. In some embodiments, as part of step 310, the flavoring agent, surfactant and other ingredients (e.g., one or more of stabilizer such as vitamin E in step 235, a cooling agent in step 237, a warming agent in step 240) are sequentially added and then mixed with water in step 315 to facilitate solubilization of these ingredients into solution. In some embodiments, as part of step 315, water is slowly added to flavoring agent, surfactant, and other ingredients with controlled speed mixing to facilitate solubilization of these components into solution.

Continuing with the non-limiting example, in some embodiments in step 310, each of the following flavoring agent and optional vitamin ingredients are sequentially added together, in the order listed, with completely mixing with the previously added ingredients before adding the next ingredient: flavoring agents, Spearmint Oil; Peppermint Oil; cooling agent, MGA; and stabilizer, Vitamin E. Next, the surfactant ingredient, Polysorbate 20, is added followed by mixing for 15 minutes or until the surfactant uniformly mixed with the previously mixed flavoring agents, cooling agent and stabilizer ingredients. Then in step 315, ambient temperature purified water equivalent to 20% of the final batch weight of the oral rinse composition added to the mixture of flavoring agents, cooling agent and stabilizer ingredients and surfactant. In some embodiments, the water is preferably added slowly enough to mitigate phase separation of the cooling agent and stabilizer (MGA and vitamin E in this example) in the aqueous dispersion.

Some embodiments of the method 300 further includes a step 320 of mixing the aqueous solution (solution “A”) and the aqueous dispersion (solution “B”) together to form an aqueous mixture (e.g., Solution “C” as referred to in FIG. 3 and elsewhere herein) such that the flavoring agent, surfactant and other ingredients are at least uniformly dispersed throughout the aqueous mixture, and in some cases dissolved in the aqueous mixture.

Some embodiments of the method 300 further includes providing one or more of the pH buffering agent (step 220; e.g., citrate and citric acid) and sweeteners (step 245; e.g., xylitol and monk fruit extract) and dissolving these components as part of step 215 to form the aqueous solution.

Some embodiments of the method 300 include a step 325 filtering (e.g., polishing filtering) the aqueous mixture (Solution C). In some embodiments the filter used in step 325 is sized (e.g., pore size in a range of about 5 to 20 micron) to facilitate the removal of colony forming units (cfu) and thereby facilitate sterilization of the aqueous mixture. However other embodiments could include alternative, or additional, sterilization steps 327 such as autoclaving, pasteurizing, exposing to ionizing radiation or similar procedures, without adding biocides to the aqueous mixture. In some embodiments, after step 325 or optional step 327 there are less than 10 cfu per 1 mL volume of the aqueous mixture (Solution C) as measured using stand procedure in accordance with the US Pharmacopeial Convention Cp 61 and California Safe Cosmetics Act M1 (e.g., Total Aerobic Microbial Count or Total Combined Yeasts and Molds Count).

The filter step 325 can also facilitate provide a clear oral rinse. However in some embodiments the oral rinse can have a cloudy or hazy appearance e.g., due to the presence of vesicles having one or more water-insoluble flavoring agents in the aqueous mixture, or, due to the aqueous mixture being an emulsion of the aqueous solution and water-insoluble flavoring agents for the aqueous dispersion.

Some embodiments of the method 300 include a step 330 of adjusting the pH of the aqueous mixture to a suitable neutral range (e.g., a pH from about 3 to about 8, and in some embodiments about 5 to about 8, and in some embodiments preferably about 6 to about 7, and in some embodiments about 6.5).

Some embodiments of the method 300 include a step 340 of adjusting the volume of the aqueous mixture, through the addition water in a quantity sufficient (quantum sufficit, Qs), that the components of the oral rinse are equal to their target concentrations.

Some embodiments of the method 300 include a step 350 of transferring the aqueous solution to a package such as a sterilized sealable glass or plastic bottle or similar containers, in volumes (e.g., about 10 to 30 milliliters) suitable for single-use oral rinse applications.

To further illustrate various features of the disclosed oral rinse composition, non-limiting example formulations of the oral rinse compositions are presented below.

Formulation 1 (Table 2; Combine Solution a with Solution B):

TABLE 2

| | Concentration | | |
|---|---|---|---|
| | (% Wt/Vol) | Concentration | |
| Component | Range | (% Wt/Vol) | Primary Purpose |
| Solution A: | | | |
| Buffer | | | |
| Sodium | — | 0.5 | Buffer |
| Citrate | | | |
| Citric acid | — | 0.05 | Buffer |
| Polyox WSK | 0.1%-5% | 0.4% | |
| N-12K | | | |
| Caffeine | 0.05%-2% | 0.2% | Energy |
| Xylitol | 0.4-0.8% | 0.8% | Sweetener |
| Ozarka | | Qs to 100% | |
| Water | | | |
| Solution B: | | | |
| Flavoring | | | |
| Peppermint | 0.01%-0.2% | 0.2% | Flavoring/Antimicrobial |
| oil | | | |
| Frescolat | 0.05%-2.0% | 0.4% | Cooling agent |
| Menthone | | | |
| Glycerin | | | |
| Acetal | | | |
| Polysorbate | 1.0%-5% | 2.% | Emulsifier/Antimicrobial |
| 20 | | | |

Formulation 2 (Table 3; Combine Solution A with Solution B):

TABLE 3

| | Concentration | | |
|---|---|---|---|
| | (% Wt/Vol) | Concentration | |
| Component | Range | (% Wt/Vol) | Primary Purpose |
| Solution A: | | | |
| Buffer | | | |
| Sodium | — | 0.53 | Buffer |
| Citrate | | | |
| Citric acid | — | 0.053 | Buffer |
| Polyox WSK | 0.1%-5% | 0.4% | |
| N-12K | | | |
| Caffeine | 0.05%-2% | 0.2% | Energy |
| Xylitol | 0.4-0.8% | 0.8% | Sweetener |
| Ozarka | | Qs to 100% | |
| Water | | | |
| Solution B: | | | |
| Flavoring | | | |
| Peppermint | 0.01%-0.2% | 0.12% | Flavoring/Antimicrobial |
| oil | | | |
| Orange oil | 0.01%-0.2% | 0.12% | Flavoring/Antimicrobial |
| Frescolat | 0.05%-2.0% | 0.4% | Cooling agent |
| Menthone | | | |
| Glycerin | | | |
| Acetal | | | |
| Polysorbate | 1.0%-5% | 2.4% | Emulsifier/Antimicrobial |
| 20 | | | |

Formulation 3 (Table 4; Combine Solution A with Solution B):

TABLE 4

| | Concentration | | |
|---|---|---|---|
| | (% Wt/Vol) | Concentration | |
| Component | Range | (% Wt/Vol) | Primary Purpose |
| Solution A: | | | |
| Buffer | | | |
| Sodium | — | 0.53 | Buffer |
| Citrate | | | |
| Citric acid | — | 0.053 | Buffer |
| Polyox WSK | 0.1%-5% | 0.4% | |
| N-12K | | | |
| Caffeine | 0.05%-2% | 0.2% | Energy |
| Xylitol | 0.4-0.8% | 0.8% | Sweetener |
| Ozarka | | Qs to 100% | |
| Water | | | |
| Solution B: | | | |
| Flavoring | | | |
| Spearmint | 0.01%-0.2% | 0.12% | Flavoring/Antimicrobial |
| Oil | | | |
| Cinnamon | 0.01%-0.2% | 0.12% | Flavoring/Antimicrobial |
| oil | | | |
| Frescolat | 0.05%-2.0% | 0.4% | Cooling agent |
| Menthone | | | |
| Glycerin | | | |
| Acetal | | | |
| Polysorbate | 1.0%-5% | 2.4% | Emulsifier/Antimicrobial |
| 20 | | | |

Formulation 4 (Table 5; Combine Solution A with Solution B):

TABLE 5

| | Concentration | | |
|---|---|---|---|
| | (% Wt/Vol) | Concentration | |
| Component | Range | (% Wt/Vol) | Primary Purpose |
| Solution A: | | | |
| Buffer | | | |
| Sodium | — | 0.53 | Buffer |
| Citrate | | | |
| Citric acid | — | 0.053 | Buffer |
| Polyox WSK | 0.1%-5% | 0.4% | |
| N-12K | | | |
| Caffeine | 0.05%-2% | 0.2-0.6% | Energy |
| Xylitol | 0.4-0.8% | 0.8-1.6% | Sweetener |
| Monk Fruit | 2-6% | 2-5.6% | Sweetener |
| Extract | | | |
| Ozarka | | Qs to 100% | |
| Water | | | |
| Solution B: | | | |
| Flavoring | | | |
| Peppermint | 0.06%-0.2% | 0.08% | Flavoring/Antimicrobial |
| oil | | | |
| Orange Oil | 0.06%-0.2% | 0.06-0.12% | Flavoring/Antimicrobial |
| Cinnamon | 0.06%-0.2% | 0.06-0.12% | Flavoring/Antimicrobial |
| oil | | | |
| Frescolat | 0.05%-2.0% | 0.4% | Cooling agent |
| Menthone | | | |
| Glycerin | | | |
| Acetal | | | |
| Polysorbate | 1.0%-5% | 2.4% | Emulsifier/Antimicrobial |
| 20 | | | |

Formulation 5 (Table 6; Combine Solution A with Solution B):

TABLE 6

| | Concentration | | |
|---|---|---|---|
| | (% Wt/Vol) | Concentration | |
| Component | Range | (% Wt/Vol) | Primary Purpose |
| Solution A: | | | |
| Buffer | | | |
| Sodium | — | 0.53 | Buffer |
| Citrate | | | |
| Citric acid | — | 0.053 | Buffer |
| Polyox WSK | 0.1%-5% | 0.4% | |
| N-12K | | | |
| Caffeine | 0.05%-2% | 0.2% | Energy |
| Xylitol | 0.4-0.8% | 0.8% | Sweetener |
| Ozarka | | Qs to 100% | |
| Water | | | |
| Solution B: | | | |
| Flavoring | | | |
| Spearmint | 0.01%-0.2% | 0.12% | Flavoring/Antimicrobial |
| oil | | | |
| Cinnamon | 0.01%-0.2% | 0.12% | Flavoring/Antimicrobial |
| oil | | | |
| Frescolat | 0.05%-2.0% | 0.4% | Cooling agent |
| Menthone | | | |
| Glycerin | | | |
| Acetal | | | |
| Polysorbate | 1.0%-5% | 2.4% | Emulsifier/Antimicrobial |
| 20 | | | |

Formulation 6 (Table 7; Combine Solution A with Solution B):

TABLE 7

| | Concentration | | |
|---|---|---|---|
| | (% Wt/Vol) | Concentration | |
| Component | Range | (% Wt/Vol) | Primary Purpose |
| Solution A: | | | |
| Buffer | | | |
| Sodium | — | 0.53 | Buffer |
| Citrate | | | |
| Citric acid | — | 0.053 | Buffer |
| Polyox WSK | 0.1%-5% | 0.4% | |
| N-12K | | | |
| Caffeine | 0.05%-2% | 0.2% | Energy |
| Xylitol | 0.4-0.8% | 0.8% | Sweetener |
| Ozarka | | Qs to 100% | |
| Water | | | |
| Solution B: | | | |
| Flavoring | | | |
| Spearmint | 0.01%-0.2% | 0.12% | Flavoring/Antimicrobial |
| oil | | | |
| Orange oil | 0.01%-0.2% | 0.12% | Flavoring/Antimicrobial |
| Frescolat | 0.05%-2.0% | 0.4% | Cooling agent |
| Menthone | | | |
| Glycerin | | | |
| Acetal | | | |
| Polysorbate | 1.0%-5% | 2.4% | Emulsifier/Antimicrobial |
| 20 | | | |

Formulation 7 (Table 8; Combine Solution A with Solution B):

TABLE 8

| | Concentration | | |
|---|---|---|---|
| | (% Wt/Vol) | Concentration | |
| Component | Range | (% Wt/Vol) | Primary Purpose |
| Solution A: | | | |
| Buffer | | | |
| Sodium | — | 0.53 | Buffer |
| Citrate | | | |
| Citric acid | — | 0.053 | Buffer |
| Polyox WSK | 0.1%-5% | 0.4% | |
| N-12K | | | |
| Caffeine | 0.05%-2% | 0.2% | Energy |
| Xylitol | 0.4-0.8% | 0.8% | Sweetener |
| Ozarka | | Qs to 100% | |
| Water | | | |
| Solution B: | | | |
| Flavoring | | | |
| Spearmint | 0.01%-0.24% | 0.24% | Flavoring/Antimicrobial |
| oil | | | |
| Frescolat | 0.05%-2.0% | 0.4% | Cooling agent |
| Menthone | | | |
| Glycerin | | | |
| Acetal | | | |
| Polysorbate | 1.0%-5% | 2.4% | Emulsifier/Antimicrobial |
| 20 | | | |

Formulation 8 (Table 9; Combine Solution A with Solution B):

TABLE 9

| | Concentration | | |
|---|---|---|---|
| | (% Wt/Vol) | Concentration | |
| Component | Range | (% Wt/Vol) | Primary Purpose |
| Solution A: | | | |
| Buffer | | | |
| Sodium | | 0.53 | Buffer |
| Citrate | | | |
| Citric acid | | 0.053 | Buffer |
| Polyox WSK | 0.1%-5% | 0.2% | |
| N-12K | | | |
| Caffeine | 0.05%-2% | 0.2%, 0.4% | Energy |
| Xylitol | 1%-15% | 4.5% | Sweetener |
| Monk Fruit | 0.1%-15% | 1.5% | Sweetener |
| Extract | | | |
| Fiji Water | | Qs to 100% | |
| Solution B: | | | |
| Flavoring | | | |
| Lemongrass | 0.01%-0.1% | 0.08% | Flavoring/Antimicrobial |
| oil | | | |
| Cardamom | 0.01%-0.1% | 0.08% | Flavoring/Antimicrobial |
| oil | | | |
| Cinnamon | 0.01%-0.1% | 0.08% | Flavoring/Antimicrobial |
| oil | | | |
| Frescolat | 0.05%-2.0% | 0.4% | Cooling agent |
| Menthone | | | |
| Glycerin | | | |
| Acetal | | | |
| Polysorbate | 1.0%-5% | 2.4% | Emulsifier/Antimicrobial |
| 20 | | | |

Formulation 9 (Table 10; Combine Solution A with Solution B):

TABLE 10

| | Concentration | | |
|---|---|---|---|
| | (% Wt/Vol) | Concentration | |
| Component | Range | (% Wt/Vol) | Primary Purpose |
| Solution A: | | | |
| Buffer | | | |
| Sodium | | 0.53 | Buffer |
| Citrate | | | |
| Citric acid | | 0.053 | Buffer |
| Polyox WSK | 0.1%-5% | 0.2% | |
| N-12K | | | |
| Caffeine | 0.05%-2% | 0.2% | Energy |
| Xylitol | 1%-15% | 9% | Sweetener |
| Monk Fruit | 0.1%-15% | 1.5% | Sweetener |
| Extract | | | |
| Fiji Water | | Qs to 100% | |
| Solution B: | | | |
| Flavoring | | | |
| Ginger oil | 0.01%-0.1% | 0.08% | Flavoring/Antimicrobial |
| Peppermint | 0.01%-1.0% | 0.16% | Flavoring/Antimicrobial |
| oil | | | |
| Frescolat | 0.05%-2.0% | 0.4% | Cooling agent |
| Menthone | | | |
| Glycerin | | | |
| Acetal | | | |
| Polysorbate | 1.0%-5% | 2.4% | Emulsifier/Antimicrobial |
| 20 | | | |

Formulation 10 (Table 11; Combine Solution A with Solution B):

TABLE 11

| | Concentration | | |
|---|---|---|---|
| | (% Wt/Vol) | Concentration | |
| Component | Range | (% Wt/Vol) | Primary Purpose |
| Solution A: | | | |
| Buffer | | | |
| Sodium | | 0.53 | Buffer |
| Citrate | | | |
| Citric acid | | 0.053 | Buffer |
| Polyox WSK | 0.1%-5% | 0.2% | |
| N-12K | | | |
| Caffeine | 0.05%-2% | 0.2% | Energy |
| Xylitol | 1%-15% | 3.5% | Sweetener |
| Monk Fruit | 0.1%-15% | 1.5% | Sweetener |
| Extract | | | |
| Fiji Water | | Qs to 100% | |
| Solution B: | | | |
| Flavoring | | | |
| Champagne | 0.01%-10.0% | 1.4% | Flavoring |
| Flavor | | | |
| Peppermint | 0.01%-1.0% | 0.05% | Flavoring/Antimicrobial |
| oil | | | |
| Lemon oil | 0.01%-1.0% | 0.05% | Flavoring/Antimicrobial |
| Menthone | 0.05%-2.0% | 0.4% | Cooling agent |
| Glycerin | | | |
| Acetal | | | |

Formulation 11 (Table 12; Combine Solution A with Solution B):

TABLE 12

| | Concentration | | |
|---|---|---|---|
| | (% Wt/Vol) | Concentration | |
| Component | Range | (% Wt/Vol) | Primary Purpose |
| Solution A: | | | |
| Buffer | | | |
| Sodium | | 0.53 | Buffer |
| Citrate | | | |
| Citric acid | | 0.053 | Buffer |
| Polyox WSK | 0.1%-5% | 0.4% | |
| N-12K | | | |
| Caffeine | 0.05%-2% | 0.2-0.6% | Energy |
| Xylitol | 1%-15% | 2% | Sweetener |
| Monk Fruit | 0.1%-6% | 2-6% | Sweetener |
| Extract | | | |
| Ozarka | | Qs to 100% | |
| Water | | | |
| Solution B: | | | |
| Flavoring | | | |
| Spearmint | 0.01%-1.0% | 0.12% | Flavoring/Antimicrobial |
| Oil | | | |
| Orange Oil | 0.01%-1.0% | 0.12% | Flavoring/Antimicrobial |
| Menthone | 0.05%-2.0% | 0.4% | Cooling agent |
| Glycerin | | | |
| Acetal | | | |
| Polysorbate | 1.0%-5% | 2.4% | Emulsifier/Antimicrobial |
| 20 | | | |

Formulation 12 (Table 13; Combine Solution A with Solution B):

TABLE 13

| | Concentration | | |
|---|---|---|---|
| | (% Wt/Vol) | Concentration | |
| Component | Range | (% Wt/Vol) | Primary Purpose |
| Solution A: | | | |
| Buffer | | | |
| Sodium | | 0.53 | Buffer |
| Citrate | | | |
| Citric acid | | 0.053 | Buffer |
| Polyox WSK | 0.1%-5% | 0.4% | |
| N-12K | | | |
| Caffeine | 0.05%-2% | 0.2% | Energy |
| Xylitol | 0.01%-2% | 0.8% | Sweetener |
| Monk Fruit | 0.1%-15% | 2% | Sweetener |
| Extract | | | |
| Ozarka | | Qs to 100% | |
| Water | | | |
| Solution B: | | | |
| Flavoring | | | |
| Spearmint | 0.01%-1.0% | 0.06% | Flavoring/Antimicrobial |
| Oil | | | |
| Orange Oil | 0.01%-1.0% | 0.06% | Flavoring/Antimicrobial |
| Cinnamon | 0.01%-1.0% | 0.06% | Flavoring/Antimicrobial |
| Oil | | | |
| Menthone | 0.05%-2.0% | 0.4% | Cooling agent |
| Glycerin | | | |
| Acetal | | | |
| Polysorbate | 1.0%-5% | 2.4% | Emulsifier/Antimicrobial |
| 20 | | | |

Formulation 13 (Table 14; Combine Solution A with Solution B):

TABLE 14

| | Concentration | | |
|---|---|---|---|
| | (% Wt/Vol) | Concentration | |
| Component | Range | (% Wt/Vol) | Primary Purpose |
| Solution A: | | | |
| Buffer | | | |
| Sodium | | 0.53 | Buffer |
| Citrate | | | |
| Citric acid | | 0.053 | Buffer |
| Polyox WSK | 0.1%-5% | 0.4% | |
| N-12K | | | |
| Caffeine | 0.05%-2% | 0.2% | Energy |
| Xylitol | 0.01%-2% | 0.8% | Sweetener |
| Monk Fruit | 0.1%-15% | 2% | Sweetener |
| Extract | | | |
| Ozarka | | Qs to 100% | |
| Water | | | |
| Solution B: | | | |
| Flavoring | | | |
| Spearmint | 0.01%-1.0% | 0.12% | Flavoring/Antimicrobial |
| Oil | | | |
| Peppermint | 0.01%-1.0% | 0.12% | Flavoring/Antimicrobial |
| Oil | | | |
| Menthone | 0.05%-2.0% | 0.4% | Cooling agent |
| Glycerin | | | |
| Acetal | | | |
| Polysorbate | 1.0%-5% | 2.4% | Emulsifier/Antimicrobial |
| 20 | | | |

Formulation 14 (Table 15; Combine Solution A with Solution B):

TABLE 15

| | Concentration | | |
|---|---|---|---|
| | (% Wt/Vol) | Concentration | |
| Component | Range | (% Wt/Vol) | Primary Purpose |
| Solution A: | | | |
| Buffer | | | |
| Sodium | | 0.53 | Buffer |
| Citrate | | | |
| Citric acid | | 0.053 | Buffer |
| Polyox WSK | 0.1%-5% | 0.4% | |
| N-12K | | | |
| Caffeine | 0.05%-2% | 0.2% | Energy |
| Xylitol | 0.01%-2% | 0.8% | Sweetener |
| Monk Fruit | 0.1%-15% | 2% | Sweetener |
| Extract | | | |
| Ozarka | | Qs to 100% | |
| Water | | | |
| Solution B: | | | |
| Flavoring | | | |
| Spearmint | 0.01%-1.0% | 0.06% | Flavoring/Antimicrobial |
| Oil | | | |
| Orange Oil | 0.01%-1.0% | 0.06% | Flavoring/Antimicrobial |
| Peppermint | 0.01%-1.0% | 0.06% | Flavoring/Antimicrobial |
| Oil | | | |
| Menthone | 0.05%-2.0% | 0.4% | Cooling agent |
| Glycerin | | | |
| Acetal | | | |
| Polysorbate | 1.0%-5% | 2.4% | Emulsifier/Antimicrobial |
| 20 | | | |

Formulation 15 (Table 16; Combine Solution A with Solution B):

TABLE 16

| | Concentration | | |
|---|---|---|---|
| | (% Wt/Vol) | Concentration | |
| Component | Range | (% Wt/Vol) | Primary Purpose |
| Solution A: | | | |
| Buffer | | | |
| Sodium | | 0.53 | Buffer |
| Citrate | | | |
| Citric acid | | 0.053 | Buffer |
| Polyox WSK | 0.1%-5% | 0.4% | |
| N-12K | | | |
| Caffeine | 0.05%-2% | 0.2-0.6% | Energy |
| Xylitol | 0.01%-2% | 0.8-1.6% | Sweetener |
| Monk Fruit | 0.1%-15% | 2-5.6% | Sweetener |
| Extract | | | |
| Ozarka | | Qs to 100% | |
| Water | | | |
| Solution B: | | | |
| Flavoring | | | |
| Spearmint | 0.01%-1.0% | 0.08-0.16% | Flavoring/Antimicrobial |
| Oil | | | |
| Peppermint | 0.01%-1.0% | 0.08-0.16% | Flavoring/Antimicrobial |
| Oil | | | |
| Menthone | 0.05%-2.0% | 0.4% | Cooling agent |
| Glycerin | | | |
| Acetal | | | |
| Polysorbate | 1.0%-5% | 2.4% | Emulsifier/Antimicrobial |
| 20 | | | |

Formulation 16 (Table 17; Combine Solution A with Solution B):

TABLE 17

| | Concentration | | |
|---|---|---|---|
| | (% Wt/Vol) | Concentration | |
| Component | Range | (% Wt/Vol) | Primary Purpose |
| Solution A: | | | |
| Buffer | | | |
| Sodium | | 0.53 | Buffer |
| Citrate | | | |
| Citric acid | | 0.053 | Buffer |
| Polyox WSK | 0.1%-5% | 0.4% | |
| N-12K | | | |
| Caffeine | 0.05%-2% | 0.2% | Energy |
| Xylitol | 0.01%-2% | 1.6% | Sweetener |
| Monk Fruit | 0.1%-15% | 4% | Sweetener |
| Extract | | | |
| Ozarka | | Qs to 100% | |
| Water | | | |
| Solution B: | | | |
| Flavoring | | | |
| Orange Oil | 0.01%-1.0% | 0.18% | Flavoring/Antimicrobial |
| Menthone | 0.05%-2.0% | 0.4% | Cooling agent |
| Glycerin | | | |
| Acetal | | | |
| Polysorbate | 1.0%-5% | 2.4% | Emulsifier/Antimicrobial |
| 20 | | | |

Formulation 17 (Table 18; Combine Solution A with Solution B):

TABLE 18

| | Concentration | | |
|---|---|---|---|
| | (% Wt/Vol) | Concentration | |
| Component | Range | (% Wt/Vol) | Primary Purpose |
| Solution A: | | | |
| Buffer | | | |
| Sodium | | 0.53 | Buffer |
| Citrate | | | |
| Citric acid | | 0.053 | Buffer |
| Polyox WSK | 0.1%-5% | 0.4% | |
| N-12K | | | |
| Caffeine | 0.05%-2% | 0.6% | Energy |
| Xylitol | 0.01%-2% | 1.6% | Sweetener |
| Monk Fruit | 0.1%-15% | 5.6% | Sweetener |
| Extract | | | |
| Ozarka | | Qs to 100% | |
| Water | | | |
| Solution B: | | | |
| Flavoring | | | |
| Grapefruit | 0.01%-1.0% | 0.18% | Flavoring/Antimicrobial |
| Oil | | | |
| Menthone | 0.05%-2.0% | 0.4% | Cooling agent |
| Glycerin | | | |
| Acetal | | | |
| Polysorbate | 1.0%-5% | 2.4% | Emulsifier/Antimicrobial |
| 20 | | | |

Formulation 18 (Table 19; Combine Solution A with Solution B):

TABLE 19

| | Concentration | | |
|---|---|---|---|
| | (% Wt/Vol) | Concentration | |
| Component | Range | (% Wt/Vol) | Primary Purpose |
| Solution A: | | | |
| Buffer | | | |
| Sodium | | 0.53 | Buffer |
| Citrate | | | |
| Citric acid | | 0.053 | Buffer |
| Polyox WSK | 0.1%-5% | 0.4% | |
| N-12K | | | |
| Caffeine | 0.05%-2% | 0.6% | Energy |
| Xylitol | 1%-15% | 3.0% | Sweetener |
| Monk Fruit | 0.1%-15% | 2.0% | Sweetener |
| Extract | | | |
| Ozarka | | Qs to 100% | |
| Water | | | |
| Solution B: | | | |
| Flavoring | | | |
| Lemongrass | 1%-20.0% | 1.8% | Flavoring |
| Fermented | | | |
| Extract | | | |
| Cinnamon | 1%-20.0% | 1.8% | Flavoring/Antimicrobial |
| Fermented | | | |
| Extract | | | |
| Cardamom | 0.01%-1.0% | 0.08% | Flavoring/Antimicrobial |
| Oil | | | |
| Menthone | 0.05%-2.0% | 0.4% | Cooling agent |
| Glycerin | | | |
| Acetal | | | |
| Polysorbate | 1.0%-5% | 1.5% | Emulsifier/Antimicrobial |
| 20 | | | |

Formulation 19 (Table 20; Combine Solution A with Solution B):

TABLE 20

| | Concentration | | |
|---|---|---|---|
| | (% Wt/Vol) | Concentration | |
| Component | Range | (% Wt/Vol) | Primary Purpose |
| Solution A: | | | |
| Buffer | | | |
| Sodium | | 0.53 | Buffer |
| Citrate | | | |
| Citric acid | | 0.053 | Buffer |
| Polyox WSK | 0.1%-5% | 0.4% | |
| N-12K | | | |
| Caffeine | 0.05%-2% | 0.6% | Energy |
| Xylitol | 1%-15% | 2.75% | Sweetener |
| Vitamin B3 | 0.006%-0.2% | 0.1% | Energy/Vitamin |
| Vitamin B6 | 0.006%-0.8% | 0.008% | Energy/Vitamin |
| Vitamin B12 | 0.00001%-0.0016% | 0.000016% | Energy/Vitamin |
| Vitamin C | 0.16%-12% | 0.5% | Energy/Vitamin |
| Vitamin D | 0.00002%-0.0005% | 0.00003% | Energy/Vitamin |
| Monk Fruit | 0.1%-15% | 4.25% | Sweetener |
| Extract | | | |
| Ozarka | | Qs to 100% | |
| Water | | | |
| Solution B: | | | |
| Flavoring | | | |
| Lemongrass | 0.01%-0.1% | 0.08% | Flavoring |
| Oil | | | |
| Cinnamon | 0.01%-0.1% | 0.08% | Flavoring/Anti- |
| Oil | | | microbial |
| Cardamom | 0.01%-0.1% | 0.08% | Flavoring/Anti- |
| Oil | | | microbial |
| Menthone | 0.05%-2.0% | 0.4% | Cooling agent |
| Glycerin | | | |
| Acetal | | | |
| Polysorbate | 1.0%-5% | 2.4% | Emulsifier/Anti- |
| 20 | | | microbial |

Formulation 20 (Table 21; Solution C) comprises a citrate buffered aqueous solution that includes Caffeine, Polyox N12K WSR, MGA, Spearmint oil, peppermint oil, and Vitamin E. Nutritional testing of formulation 20 indicated 4 calories per 10 mL of the formulation. In some embodiments, of Formulation 20, about 25 percent more of MGA than listed in the Table 21 is included to account for losses when the formulation is autoclaved in a sterilization step. In some embodiment of Formulation 20, the concentration of Polysorbate 20 is 3.0% Wt/vol to accommodate for increases in oil soluble components for autoclave processing. In some embodiment of Formulation 20, about 20 to 30 percent more of Polyox N12K Water Soluble Resin (WSR) than listed in the Table 21 is included to account for losses when the formulation is autoclaved in a sterilization step.

TABLE 21

| | Concentration | |
|---|---|---|
| Component | (% Wt/Vol) | Primary Purpose |
| Sodium Citrate | 0.53 | |
| Citric Acid | 0.053 | Buffer |
| Polyox N12K Water | 0.48 | Buffer |
| Soluble Resin (WSR) | | |
| Caffeine | 0.6 | |
| Frescolat Menthone | 0.4 | Energy |
| Glycerin Acetal (MGA) | | |
| Polysorbate 20 | 2.4 | Sweetener |
| Spearmint Oil | 0.16 | Energy/Vitamin |
| Peppermint Oil | 0.08 | Energy/Vitamin |
| Vitamin E | 0.1 | Energy/Vitamin |
| (dl-alpha-tocopherol) | | |
| Xylitol | 1.6 | Energy/Vitamin |
| Monk Fruit Extract | 4.0 | Energy/Vitamin |
| Purified Water | Qs to 100 | Sweetener |
| | w/v % | |
| Final pH | 6.5 | |

Those skilled in the pertinent arts to which this application relates will appreciate that other and further additions, deletions, substitutions and modifications may be made to the described embodiments.

What is claimed:

1. An oral rinse composition, comprising:
an aqueous solution that includes a high molecular weight polymer and an energy supplement that stimulates central nervous system activity.

2. The composition as recited in claim 1, wherein the energy supplement includes caffeine having a concentration in the aqueous solution in a range from about 0.001 to about 2% Wt/Vol.

3. The composition as recited in claim 2, wherein the caffeine concentration in the aqueous solution is in a range from about 0.2 to about 1% Wt/Vol.

4. The composition as recited in claim 1, wherein the energy supplement includes one or more of an amino acid, a phospholipid, a vitamin, a cellular metabolite, or an acetyl choline enhancer.

5. The composition as recited in claim 1, wherein the high molecular weight polymer has a molecular weight of at least about 100,000 grams per mole and less than about 7 million grams per mole.

6. The composition as recited in claim 5, wherein the molecular weight is in a range from about 1 to about 3 million grams per mole.

7. The composition as recited in claim 1, wherein the oral rinse composition at room temperature and zero shear, has a viscosity in the range from about 4 cp to about 10 cp.

8. The composition as recited in claim 7, wherein at a shear rate in a range from about 50 sec⁻¹ to about 1000 sec⁻¹ the viscosity of the oral rinse composition drops by at least about 10 percent within about 30 seconds, and, the viscosity of the oral rinse composition returns to the viscosity at zero shear value upon terminating of the shear rate in the range.

9. The composition as recited in claim 1, wherein the high molecular weight polymer includes a poly (ethylene oxide) polymer.

10. The composition as recited in claim 1, wherein the high molecular weight polymer has a concentration in the aqueous solution that is in a range from about 0.01 to about 10 wt/vol %.

11. The composition as recited in claim 1, wherein the high molecular weight polymer can include a hydrophilic polymer.

12. The composition as recited in claim 1, wherein the oral rinse composition includes a buffering agent and has a pH in a range from about 3 to about 8.

13. The composition as recited in claim 1, wherein the oral rinse composition includes one or more of a surfactant, a skin and mucosal penetration enhancer, a stabilizer, a cooling agent, a warming agent or a flavoring agent.

14. The composition as recited in claim 1, wherein the oral rinse composition is an emulsion of the aqueous solution and a water-insoluble one of a flavoring agent, cooling agent, warming agent or stabilizer.

15. An oral rinse composition, comprising:
an aqueous solution including:
caffeine;
sodium citrate and citric acid;
menthyl glycerin acetal; polysorbate 20;
spearmint oil;
peppermint oil;
vitamin E;
Xylitol;
monk fruit extract; and
a poly(ethylene oxide) polymer having a molecular weight of about 1 million and a pH equal to about 6.5.

16. A method of manufacturing an oral rinse composition, comprising:
preparing an aqueous solution, including:
providing a high molecular weight polymer;
providing an energy supplement that stimulates central nervous system activity; and
dissolving the high molecular weight polymer and the energy supplement in water to form an aqueous solution.

17. The method as recited in claim 16, further including:
preparing an aqueous dispersion, including:
providing a flavoring agent;
providing a surfactant; and
mixing the flavoring agent and surfactant together in water such that the flavoring agent and surfactant are uniformly dispersed in the water; and
mixing the aqueous solution and the aqueous dispersion together to form an aqueous mixture such that the flavoring agent and surfactant are uniformly dispersed throughout the aqueous mixture.

18. The method as recited in claim 16, further including adding acid or base to adjust the aqueous solution to provide a pH in a range from about 3 to about 8.

19. The method as recited in claim 16, further including sterilizing the aqueous solution.

20. The method as recited in claim 16 further including filtering the aqueous solution to provide a clear oral rinse composition.
